(19) United States
(12) Patent Application Publication
(10) Pub. No.: US 2024/0150182 A1
(43) Pub. Date: May 9, 2024

(54) PROCESS OF REACTING SILICONE WITH HYDROTHERMAL OR SUPERCRITICAL WATER

(71) Applicant: Wacker Chemie AG, Munich (DE)

(72) Inventors: Markus ULBRICH, Munich (DE); Jan TILLMANN, Munich (DE); Harald VOIT, Reischach (DE)

(73) Assignee: Wacker Chemie AG, Munich (DE)

(21) Appl. No.: 18/283,028

(22) PCT Filed: Apr. 7, 2021
(86) PCT No.: PCT/EP2021/059103

§ 371 Date: Sep. 20, 2023

(51) Int. Cl. C01B 32/907; C01B 33/021; C07C 1/32; C07F 7/08

(52) U.S. Cl. CPC C01B 32/907; C01B 33/021; C07C 1/321; C07F 7/0801

(57) ABSTRACT

A process for reacting silicone with hydrothermal water is at temperatures from 140° C. or supercritical water. A solids mixture containing silicon is formed.

The invention relates to a process for reacting silicone with hydrothermal or supercritical water.

Silicone wastes are currently sent for incineration. They have poor combustibility, form dusts which burden the incineration, and require high airflows/combustion chamber temperatures for reaction. The dust in this operation is obtained together with the ash as a toxic biproduct and requires disposal, which is costly and inconvenient.

The use of hydrothermal water as a chemical reagent was studied in 1913 by Friedrich Bergius as part of the gasification of coal and was identified there as a key component for natural carbonization. In the hydrothermal range, the dielectric constant of the water drops to a level that allows apolar substances to be dissolved. As a result of the high temperature and the high pressure, a complex reaction network operates for lignocellulose, i.e., organic substance. The lignocellulose here is broken down by hydrolysis of C—O bonds and solid-state reactions and/or intramolecular rearrangements and elimination reactions of the lignin and is converted into a C-rich substance. Depending on temperature, a solid, an oil or gas is obtained. In the area of biomass utilization, hydrothermal processes have in recent years come strongly into the focus of research.

Kenichiro Itami, Koji Terakawa, Jun-ichi Yoshida, and Okitsugu Kajimoto, in “The Carbon-Silicon Bond Cleavage of Organosilicon Compounds in Supercritical Water” The Chemical Society of Japan Bull. Chem. Soc. Jpn., 77, 2071-2080 (2004), describe the cleavage of Si—C bonds in silanes with supercritical water.

The invention provides a process wherein silicone is reacted with hydrothermal water at temperatures from 140° C. or supercritical water to give a solids mixture containing silicon.

Water referred to as hydrothermal in this context is non-supercritical water at temperatures above 100° C. which, as a result of pressure greater than or equal to the vapor pressure associated with the temperature, is present in the liquid state. Supercritical water refers to water at temperatures and pressures higher than or equal to the critical point at 374.12° C. and 22.1 MPa.

Surprisingly, success has been achieved in cleaving not only the Si—C bonds but also the Si—O bonds in silicones through hydrothermal reaction, and in converting the resultant intermediates into a solid, similar to precipitated silica. The hydrothermal cleavage was hitherto known only for Si—C bonds.

The process affords the possibility of reacting liquid and solid silicones in the liquid phase or supercritical phase. The reaction mixture of water and silicone/siloxane produces a hydrophobic, non-toxic solids mixture which can subsequently either be disposed of unproblematically or, owing to the hydrophobic properties, used as filling material. This solids mixture may also be passed to raw silicone production, signifying the closing of the silicon->methylchlorosilanes->siloxane->solids mixture->silicon cycle. The carbon in the solid contributes to reducing the amount of coal needed for raw silicon production. This solids mixture can also be used as a filler for cement mixtures.

The term “silicone” encompasses oligomeric or polymeric organosiloxanes in which silicon atoms are connected via oxygen atoms and in which at least some of the silicon atoms carry one or more organic constituents, and also compositions comprising organosiloxanes.

The silicones contain preferably at most 1 wt %, more preferably at most 0.1 wt %, more particularly at most 0.01 wt % of halogens, especially chlorine.

Compositions containing organosiloxanes may as well as organosiloxanes contain, for example, fillers, catalysts, binders and pigments. Fillers are, for example, fumed and/or precipitated silica, chalk and quartz.

The process may be carried out batchwise or continuously.

The solids mixture obtained in the process contains preferably 25 to 50 wt %, more preferably 30 to 45 wt %, more particularly 31 to 40 wt % of silicon. The solids mixture contains preferably 0 to 65 wt %, more preferably 8 to 40 wt %, more particularly at least 15 to 35 wt % of carbon.

The carbon and oxygen fractions in the solid here are directly dependent on reaction temperature and on the composition of the starting materials. Higher temperatures lead to lower carbon contents and higher oxygen contents.

In one particular embodiment, a gas mixture is formed. The gas mixture contains preferably methane. Up to 400 g of methane may be produced per kg of silicone used. Preferably 10 to 300 g of methane are produced per kg of silicone used.

In one particular embodiment, the process also produces a liquid phase. The liquid phase contains water, biproducts of the reaction, and impurities introduced by the reactants. Depending on the operating regime, a part or the entire liquid phase may be used again as a reactant, in order to minimize wastewater volumes, for example.

Besides methane, variable proportions are formed of carbon monoxide, carbon dioxide, higher hydrocarbons, hydrogen and water. The composition of the gas mixture is dependent on pressure and temperature. With higher temperature under higher pressure, the proportion of methane in the gas mixture can be increased.

The preferred temperature in the process is 160° C. to 700° C., more particularly 200° C. to 400° C. and very preferably 250° C.-390° C. The preferred pressure in the process is from 10 to 400 bar, more preferably 20 to 320 bar, more particularly 50 to 280 bar, with the lower limit on the pressure being taken in each case to be the vapor pressure of the reaction mixture that is associated with the reaction temperature. The preferred residence time in the process is 1 minute to 24 hours, more preferably 5 minutes to 10 hours, more particularly 10 minutes to 1 hour.

In one preferred embodiment, the reaction mixture is depressurized to atmospheric pressure after the end of the reaction and the resultant vapor is utilized for heat recovery and/or preheating of the reactants. As a result, the heat can be recovered to a large extent.

The examples which follow serve for further elucidation of the invention described here.

The following analytical methods and instruments are used for characterization:

The elemental contents reported in the examples are measured using an energy-dispersive x-ray spectrometer (EDX). The EDX analyses were carried out using a Zeiss Ultra 55 scanning electron microscope and an Oxford X-Max 80N energy-dispersive x-ray spectrometer. Before the analysis, the samples were vapor-coated with carbon to prevent charging phenomena, using a Safematic Compact Coating Unit 010/HV. The gas phase is analyzed via mass spectrometry (ThermoStar™ GSD 320 T2 with iridium cathode).

The following materials and apparatuses are used in conducting experimental examples:

The autoclave used in example 1 consists of a cylindrical lower part (beaker) and a cover with multiple ports (for gas withdrawal, temperature measurement and pressure measurement, for example). The volume of the autoclave is 594 ml. The autoclave is heated electrically. The stirrer used is a four-blade inclined-blade stirrer having a diameter of 5 cm.

The silicone used in example 1 is ELASTOSIL® LR 7665 from WACKER CHEMIE AG, Munich, fully cured according to instruction. The water used is distilled water.

In the examples below, in each case unless otherwise indicated, all quantities and percentages are based on weight, all pressures are 0.10 MPa (abs.), and all temperatures are 20° C.

### EXAMPLE 1

An autoclave is charged with an amount of 30 g of finely chopped (cubes with an edge length of 0.5 cm) silicone and 150 ml of water, and closed. Over the course of 120 minutes, the autoclave is heated to a temperature of 350° C., and the temperature is held for 90 minutes. In the course of the experiment, the pressure climbs to 160 bar. Over the course of 12 hours, the autoclave cools to room temperature. After cooling of the reactor, the sample is taken for gas analysis. The gas space of the autoclave is subsequently purged with nitrogen and the autoclave is opened.

A clear liquid and a white or slightly grey solid are obtained. After the liquid has been removed and the solid has been dried, 21 g of solid are obtained.

3 experiments were conducted. The results of the EDX analyses of the solids mixture are listed in table 1. MV denotes mean value.

TABLE 1

| Experiment no. | 1 | 2 | 3 | Max | Min | MV |
|---|---|---|---|---|---|---|
| C (wt %) | 23.96 | 34.09 | 29.64 | 34.09 | 23.96 | 29.23 |
| O (wt %) | 41.35 | 32.64 | 38.46 | 41.35 | 32.64 | 37.48 |
| Na (wt %) | 0.00 | 0.21 | 0.29 | 0.29 | 0.00 | 0.17 |
| Si (wt %) | 34.70 | 33.05 | 31.61 | 34.70 | 31.61 | 33.12 |
| Total (wt %) | 100.01 | 99.99 | 100.00 | | | |

### EXAMPLE 2

1000 kg of solid silicone wastes, consisting of silicone rubber in plastic containers, are comminuted, containers included, and reacted in a stirred tank with a size of 3 m 3 together with 1500 kg of water at 350° C. and 160 bar for 2 h. Two tanks are operated in parallel. The preheating of the one tank is realized by cooling of the other tank, via heat recovery; the rest of the heat required is supplied externally. The products of the reaction are a solids/liquid mixture and a gas phase. The solids/liquid mixture is filtered; the solid obtained has the composition from table Table 1. According to present knowledge of the hydrothermal carbonization, any plastic containers added are reacted to give carbon-rich particulate solids. The liquid phase contains biproducts of the reaction and, depending on the degree of contamination, may be used for the further reaction of silicone wastes. As well as water, the gas phase contains volatile biproducts, possibly including methane, depending on reaction regime and starting products.

The solids are passed on for profitable utilization from example 3.

### EXAMPLE 3

Profitable utilization of the solids from example 1 and example 2 for producing metallurgical silicon.

The solids obtained in examples 1 and 2 are pressed to give pellets and supplied as a starting material for raw silicone production. In that process, the silicon is reduced to raw silicon.

1-8. (canceled)

9. A process for reacting silicone with hydrothermal water at temperatures from 140° C. or supercritical water to give a solids mixture containing silicon,
wherein the term “silicone” encompasses oligomeric or polymeric organosiloxanes in which silicon atoms are bonded via oxygen atoms and in which at least some of the silicon atoms carry one or more organic substituents, and also compositions comprising organosiloxanes.

10. The process as claimed in claim 9, wherein the silicones contain at most 1 wt % of halogens.

11. The process as claimed in claim 9, wherein the solids mixture contains 25 to 50 wt % of silicon.

12. The process as claimed in claim 9, wherein a methane-containing gas mixture is produced.

13. The process as claimed in claim 9, wherein the temperature is 160° C. to 700° C.

14. The process as claimed in claim 9, wherein the pressure is 10 to 400 bar.

15. The process as claimed in claim 9, wherein the solids mixture produced is supplied to raw silicon production.
